(19) United States
(12) Patent Application Publication
(10) Pub. No.: US 2022/0062532 A1
(43) Pub. Date: Mar. 3, 2022

(54) INTRAVENOUS LINE HOLDER

(71) Applicant: IV-GLOVE INC., Toronto (CA)

(72) Inventors: Manny BARON, Corunna, Ontario (CA); Ross HARRIS, Corunna, Ontario (CA); Kareem SHERIF, Toronto, Ontario (CA)

(21) Appl. No.: 17/414,750

(22) PCT Filed: Dec. 16, 2019
(86) PCT No.: PCT/CA2019/051825

§ 371 Date: Jun. 16, 2021

Related U.S. Application Data
(60) Provisional application No. 62/780,293, filed on Dec. 16, 2018.

(51) Int. Cl. A61M 5/14; A61M 25/02

(52) U.S. Cl. CPC A61M 5/1418; A61M 25/02; A61M 2209/088; A61M 2025/028; A61M 2025/024; A61M 2025/0213; A61M 2025/0206

(57) ABSTRACT

Described is an apparatus for supporting an intravenous line. The apparatus includes an intravenous line housing defining an intravenous line guide. The intravenous line guide is configured to hold the intravenous line and direct the intravenous line along a reversing path and to a substantially proximally-directed entry angle for entry into a dorsal surface of a user's hand. The apparatus further includes a glove that is secured to the intravenous line housing, where the glove is configured to hold the intravenous line housing to the user's hand.

### FIELD OF THE INVENTION

The present specification relates generally to medical devices, and specifically to an apparatus for holding an intravenous line.

### BACKGROUND OF THE INVENTION

Intravenous lines are commonly used to introduce fluid into a patient. Intravenous lines often include an intravenous tube and an intravenous tip; the intravenous tube provided to carry a fluid from a source to the tip. The tip is commonly inserted through the skin of a patient and into a vein.

When the tip of an intravenous line is inserted through the skin it is often desirable to secure the line to the anatomy of the patient. Movement of the line can contribute to a variety of issues, such as removal of the tip from the vein or subcutaneous damage caused by movement of the tip.

A variety of fasteners have been employed to hold intravenous lines. For example, adhesive tape is commonly used to secure a line to a patient. Various straps and gloves have also been developed, many of which perform dual roles in securing an intravenous line and shielding the insertion point from contamination. However, these creations have many limitations, and none provide an ideal solution.

Accordingly, there remains a need for improvements in the art.

### SUMMARY OF THE INVENTION

In an embodiment of the present invention, there is provided an apparatus for supporting an intravenous line, comprising an intravenous line housing defining an intravenous line guide, the intravenous line guide configured to hold the intravenous line and direct the intravenous line along a reversing path and to a substantially proximally-directed entry angle for entry into a dorsal surface of a user's hand; and a glove secured to the intravenous line housing, the glove configured to hold the intravenous line housing to the user's hand.

In an embodiment of the present invention, there is provided a glove for holding an intravenous line, comprising a fabric panel configured to be releasably fastened to a user's hand; and a set of clips secured to the fabric panel, the set of clips configured to hold the intravenous line and direct the intravenous line along a reversing path and to a substantially proximally-directed entry into a dorsal surface of the user's hand.

Other aspects and features according to the present application will become apparent to those ordinarily skilled in the art upon review of the following description of embodiments of the invention in conjunction with the accompanying figures.

### BRIEF DESCRIPTION OF THE DRAWINGS

The principles of the invention may better be understood with reference to the accompanying figures provided by way of illustration of an exemplary embodiment, or embodiments, incorporating principles and aspects of the present invention, and in which:

FIG. 1 is a top plan view of an apparatus for holding an intravenous line, according to an embodiment, mounted on a user's hand;

FIG. 2 is a top plan view of the apparatus of FIG. 1, mounted on a user's hand and with the outer cover removed and with an intravenous line affixed;

FIG. 3 is a top plan view of the apparatus of FIG. 1, mounted on a user's hand and with the outer cover removed;

FIG. 4 is a bottom plan view of the apparatus of FIG. 1, mounted on a user's hand;

FIG. 5 is a right side elevation view of the apparatus of FIG. 1, mounted on a user's hand;

FIG. 6 is a left side elevation view of the apparatus of FIG. 1, mounted on a user's hand;

FIG. 7 is a top plan view of the apparatus of FIG. 1, opened and with the outer cover affixed;

FIG. 8 is a top plan view of the apparatus of FIG. 1, opened and with the outer cover removed;

FIG. 9 is a top plan view of an apparatus for holding an intravenous line, according to a second embodiment, mounted on a user's hand and with an intravenous line affixed;

FIG. 10 is a right side perspective view of the apparatus of FIG. 9, mounted on a user's hand;

FIG. 11 is a left side perspective view of the apparatus of FIG. 9, mounted on a user's hand;

FIG. 12 is a top plan view of the apparatus of FIG. 9, mounted on a user's hand;

FIG. 13 is a bottom plan view of the apparatus of FIG. 9, mounted on a user's hand;

FIG. 14 is a top plan view of the apparatus of FIG. 9, opened and with an opaque cover affixed;

FIG. 15 is a bottom perspective view of the apparatus of FIG. 9, closed but unmounted;

FIG. 16 is a top perspective view of the apparatus of FIG. 9, closed but unmounted and with a clear cover affixed;

FIGS. 17a, 17b and 17c show an apparatus for holding an intravenous line according to a third embodiment mounted on a user's hand and with an intravenous line affixed from a top plan view, bottom plan view and a right elevational view, respectively; and

FIGS. 18a, 18b and 18c show an apparatus for holding an intravenous line according to a fourth embodiment mounted on a user's hand and with an intravenous line affixed from a top plan view, bottom plan view and a right elevational view, respectively.

### DETAILED DESCRIPTION OF THE EMBODIMENTS

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order more clearly to depict certain features of the invention.

An aspect of this description relates to an apparatus for supporting an intravenous line inserted into a user's hand. An aspect of this description relates to an apparatus which incorporates a guide structure designed to hold an intravenous line and guide the intravenous line along a predefined path into a patient's hand.

An aspect of this description relates to a support apparatus which incorporates a guide structure designed to hold an intravenous line, and to guide the intravenous line along a reversing path and to direct the intravenous line to a substantially proximally directed entry. In some embodiments, an intravenous line guide is a clip structure defined by a set of projections of a housing. In further embodiments, the intravenous line guide may be recessed into a groove in the housing and the intravenous line may be held by a friction fit. Optionally, the intravenous line guide may be further retained in such embodiments using one or more straps with accompanying snaps and eyelets in case the intravenous line is pulled out of the groove.

An aspect of this description relates to a glove having an intravenous line guide which defines a curved path for an intravenous line. In some embodiments, the curved path includes an s-shaped path.

An aspect of this description relates to an apparatus for holding an intravenous line and guiding the line along a predetermined path. An aspect of this description relates to an apparatus which includes a glove structure and a housing defining an intravenous line guide. In some embodiments, the glove structure is a rigid structure, such as a cast-style glove formed of rigid plastic or similar material. In some embodiments, the glove structure is semi-rigid, such as a glove formed of plastic plates coupled at flexible joints. In preferred embodiments, the glove structure is a flexible structure, such as a system of fabric straps. In some embodiments, the intravenous line guide structure is a clip or a set of clips provided to hold an intravenous line in a predetermined position. In some embodiments, the intravenous line guide is a groove recessed into the housing provided to hold an intravenous line. In some embodiments, the intravenous line guide is a combination of a clip or set of clips and a cover or cap, which cooperate to hold an intravenous line in a predetermined position.

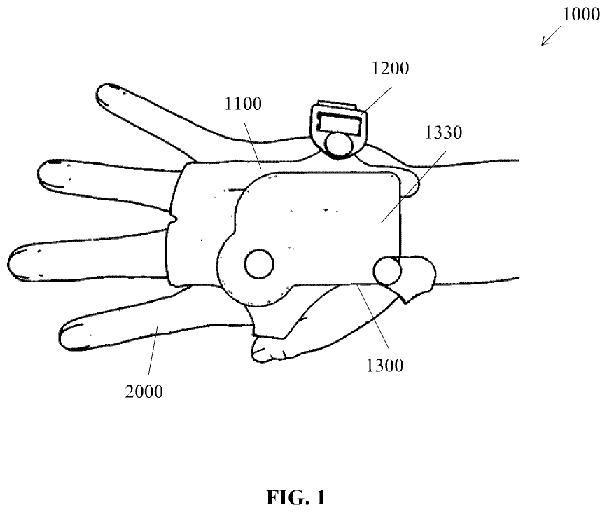

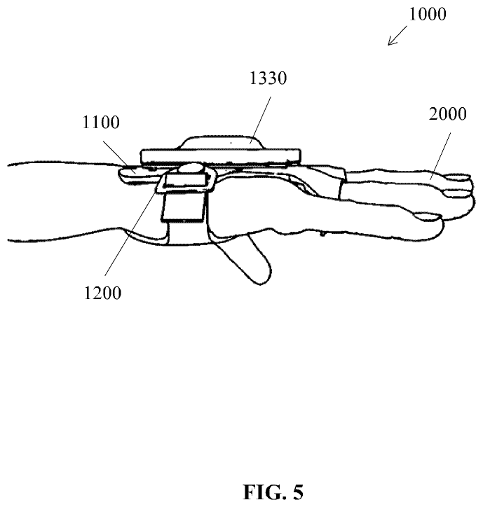

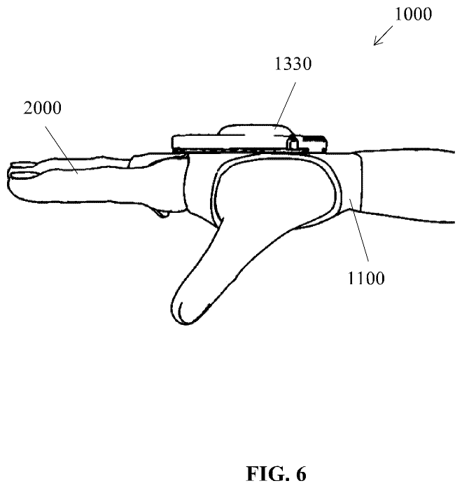

An embodiment of an apparatus for holding an intravenous line is depicted in FIGS. 1 to 6. Apparatus 1000 includes a fabric glove 1100, a buckle 1200 and a housing 1300.

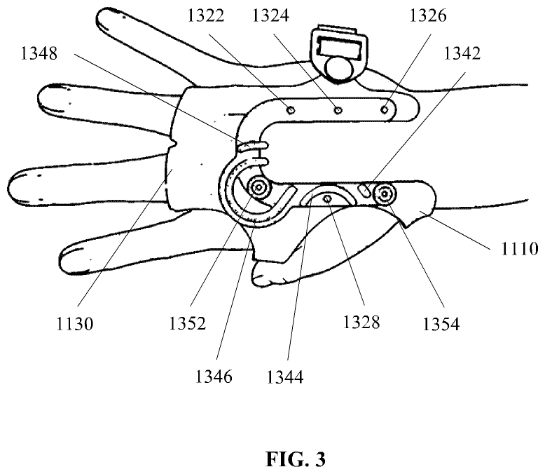

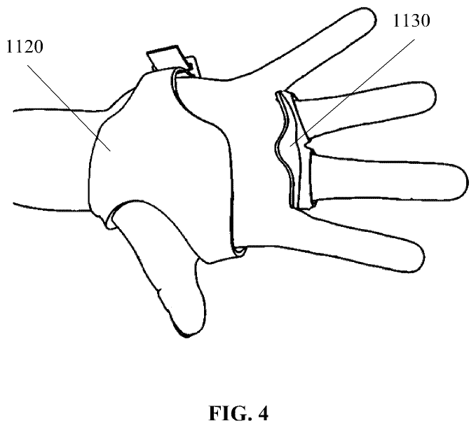

Apparatus 1000 is shown secured to a hand 2000; the fabric glove 1100 is a continuous panel which is releasably secured via buckle 1200 to itself around hand 2000. Glove 1100 of apparatus 1000 forms a mounting panel 1110, a palm strap 1120, and a pair of finger loops 1130, shown particularly in FIGS. 3 and 4; mounting panel 1110 is provided to hold housing 1300, palm strap 1120 is provided to extend under the user's hand and join to the mounting panel 1110 via buckle 1200, and the forward finger loops 1130 are provided to fit over the fingers of a user.

Housing 1300 is provided to receive and hold a length of intravenous line, and to house the insertion point of an intravenous line into a dorsal surface of a user's hand. Housing 1300 is mounted on mounting panel 1110 of glove 1100 and configured to cover a portion of the dorsal surface of the user's hand.

Housing 1300 includes a footing 1310 secured to glove 1100 by a set of fasteners 1320. Housing 1300 also includes an outer cover 1330 secured to the footing 1310 by way of a combination of a friction overlay of footing 1310 and a set of fasteners 1350. Footing 1310 defines a set of clip projections 1340, together the set of clip projections 1340 define an intravenous line guide for holding an intravenous line. The outer cover 1330 also cooperates with the set of clip projections 1340 to hold the intravenous line in a predefined position. In some embodiments, a cover is a ventilated cover.

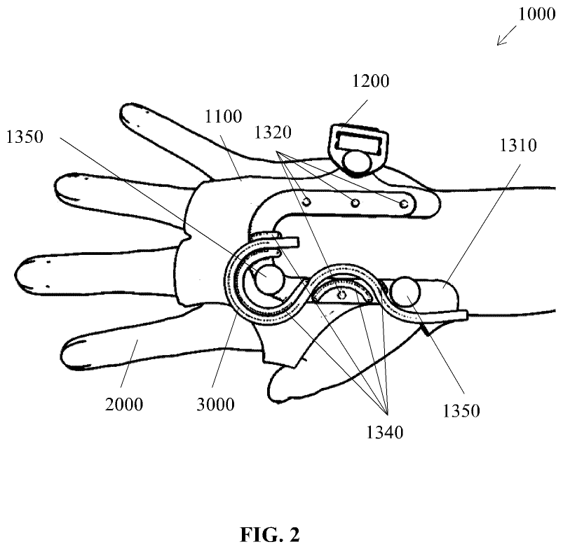

Apparatus 1000 is shown in FIG. 1 with outer cover 1330 of housing 1300 affixed. Apparatus 1000 is shown in FIGS. 2 and 3 with outer cover 1330 removed.

The set of clip projections 1340 of apparatus 1000 is a set of four projections. A first projection 1342 and a second projection 1344 define a beginning of an intravenous tube guide while a third projection 1346 and a fourth projection 1348 define an end of the intravenous tube guide.

Fasteners 1320 are a set of four pins, three on the right side 1322, 1324, and 1326 and one on the left 1328, bonded or adhered to glove 1100 and holding footing 1310 via an interference fit. Fasteners 1350 are a set of two pins, a distal pin 1352 and a proximal pin 1354, bonded or adhered to glove 1100 and designed so that cover 1330 may be received between a base portion of each pin and a cap portion, with the base and cap portions held to one another via an interference fit. While the embodiment shown uses an interference fit, in various embodiments the apparatus may have components secured to one another in a variety of ways, such as direct stitching, adhesion, hook-and-loop fasteners, and moulding.

Glove 1100 of apparatus 1000 defines a dorsal opening which is provided to leave the back of a user's hand uncovered by glove 1100 in an area where an intravenous line is likely to be entered into the hand. Footing 1310 likewise defines a dorsal opening to leave the back of a user's hand uncovered. The dorsal opening is then covered by cover 1330 which sits over footing 1310 and the intravenous line guide defined by projections 1340. Cover 1330 is clear so that an entry site of the intravenous line 3000 may be observed, such as by a user or healthcare provider, without removing the cover 1330.

The use of a clear cover also allows for the observation of the part of the intravenous tube which is covered by the cover. Cover 1330 cooperates with the set of projections 1340, particularly projection 1346, to hold the intravenous line 3000 in position. Cover 1330 also includes a notch or opening to permit the entry of the intravenous line 3000 into the housing. Housing 1300 is configured to receive the intravenous line 3000 from a left side of the housing, guide it along an s-shaped and reversing path, and direct it to a substantially proximally directed entry angle.

Housing 1300 defines an intravenous line guide using a combination of the clip projections 1340 and the cover 1330. As depicted, the intravenous line guide is an ‘s-clip’ which defines an exaggerated s-shaped guide; accepting an intravenous line from a left side of the housing, guiding it around a first curve and then a reversing second curve to an entry point.

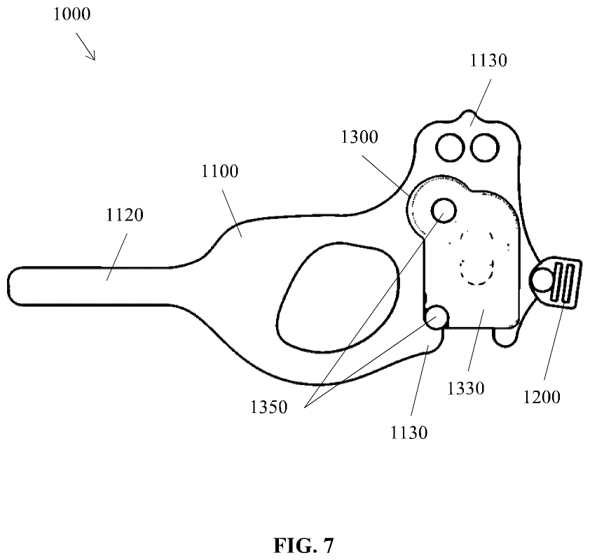

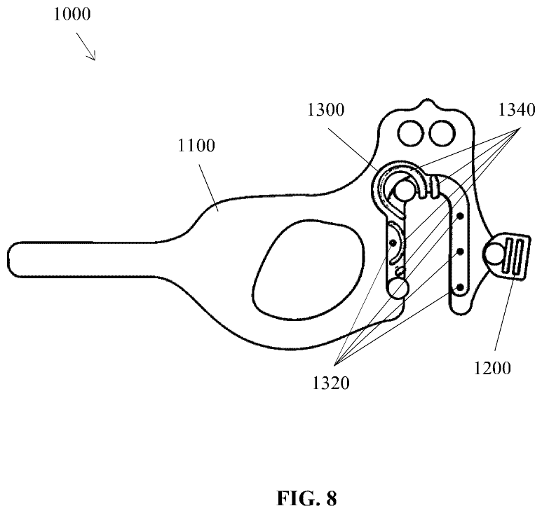

Apparatus 1000 is shown in FIGS. 7 and 8 as well. In FIGS. 7 and 8 apparatus 1000 is in an opened positioning in which the palm strap is released from ladder lock buckle 1200 and glove 1100 is spread out. As may be seen in FIGS. 7 and 8, glove 1100 is a glove blank on which buckle 1200 and housing 1300 are mounted to form apparatus 1000. Components of apparatus 1000 are designed for ease of manufacturing, transportation and storage, and use. Various materials may be used in the construction. For example, in some embodiments a housing, or elements of a housing, are made of plastic, such as moulded from a firm plastic. In some embodiments, a glove is a fabric glove, such as a fabric glove made of a microfiber or a scuba-like material such as a neoprene fabric.

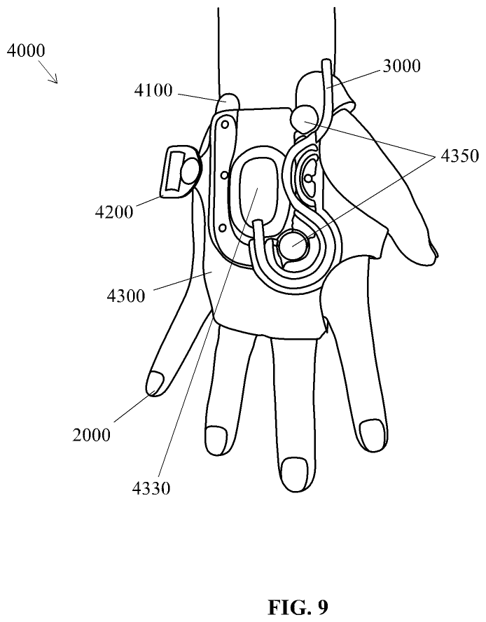

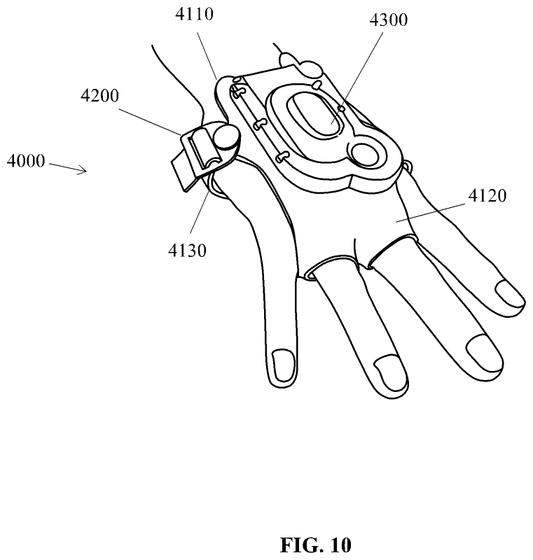

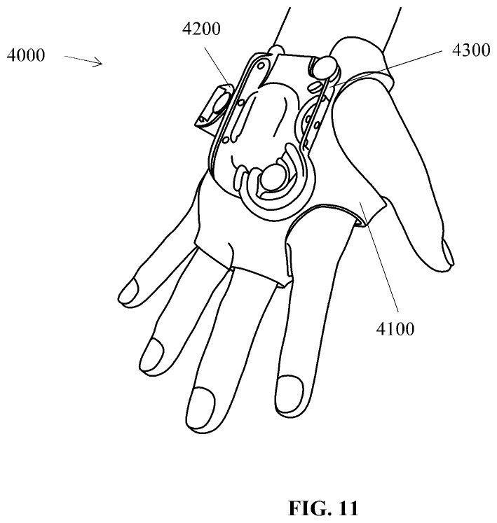

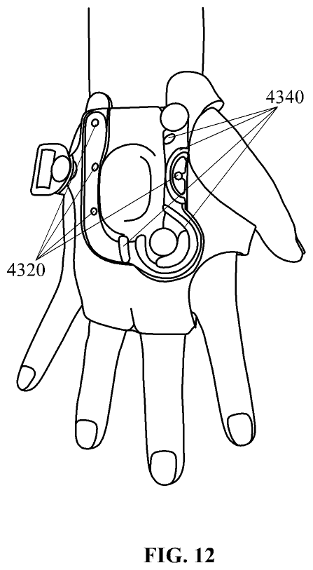

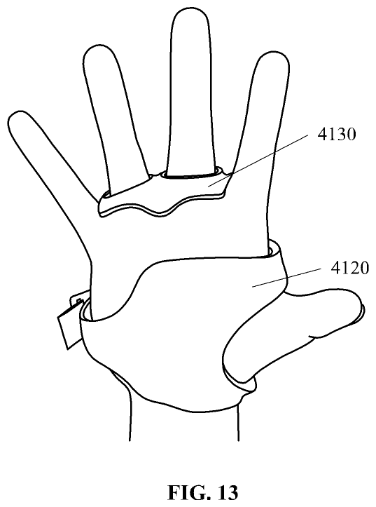

A second embodiment is shown in FIGS. 9 to 13. Apparatus 4000 is shown on hand 2000 and holding intravenous line 3000. Apparatus 4000 includes a fabric glove 4100, a buckle 4200 and a housing 4300.

The fabric glove 4100 is a continuous panel which is secured via buckle 4200 to itself around hand 2000. Glove 4100 of apparatus 4000 forms a mounting panel 4110, a palm strap 4120, and a pair of finger loops 4130. Mounting panel 4110 is provided to hold housing 4300, palm strap 4120 is provided to extend under the user's hand and join to the mounting panel 4110 via buckle 4200, and the forward finger loops 4130 are provided to fit over the fingers of a user.

Housing 4300 is provided to house the insertion point of an intravenous line. Housing 4300 is mounted on glove 4100 on a dorsal surface of hand 2000. Housing 4300 is designed to receive an intravenous line for insertion into the dorsal surface of hand 2000.

Housing 4300 includes a footing 4310 secured to glove 4100 by a set of fasteners 4320. Housing 4300 also includes an upper cover 4330 secured to the footing 4310 by way of a combination of a friction overlay of footing 4310 and a set of fasteners 4350. Footing 4310 defines a set of clip projections 4340, together the set of clip projections 4340 define an intravenous line guide for holding an intravenous line. Fasteners may be held to panel 4110 in a variety of ways, such as via adhesive, mechanical clips, or hook-and-loop components.

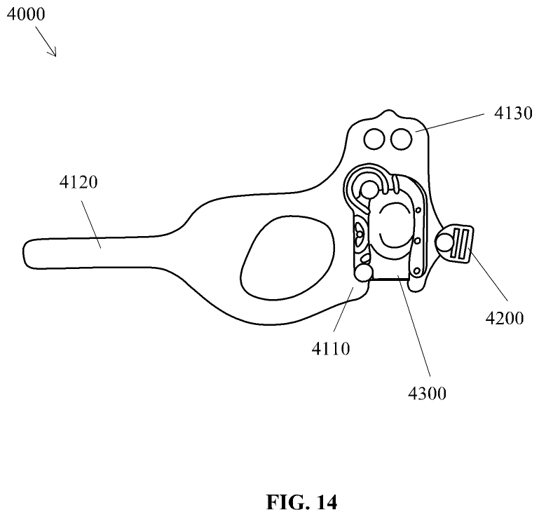

Cover 4330 may be clear or opaque; it is shown in FIGS. 9 to 12 and 16 as a clear cover and in FIG. 14 as an opaque cover. Cover 4330 may also be interchangeable, such as to allow a clear cover to be swapped for an opaque cover.

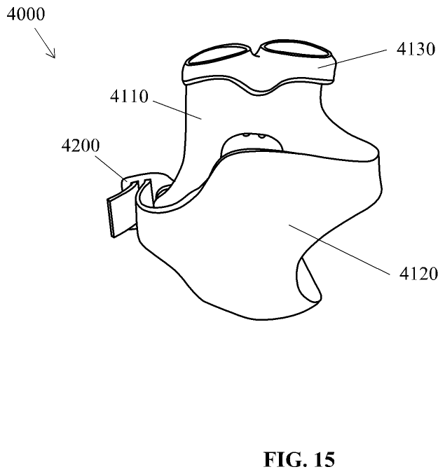

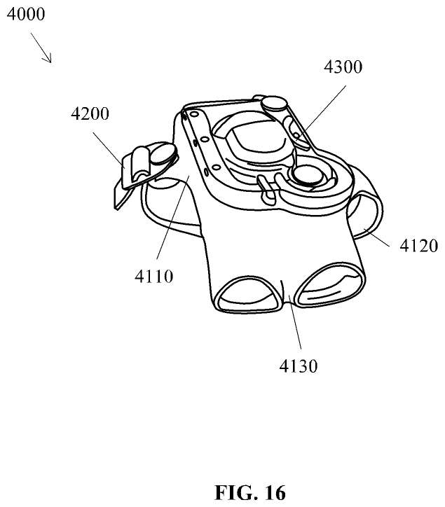

Apparatus 4000 is also shown in FIGS. 14 to 16. In FIG. 14 apparatus 4000 is in an opened positioning in which the palm strap is released from ladder lock buckle 4200 and glove 4100 is spread out. In FIGS. 15 and 16 apparatus 4000 is in a closed positioning in which the palm strap is secured to ladder lock buckle 4200 but is empty of a user's hand.

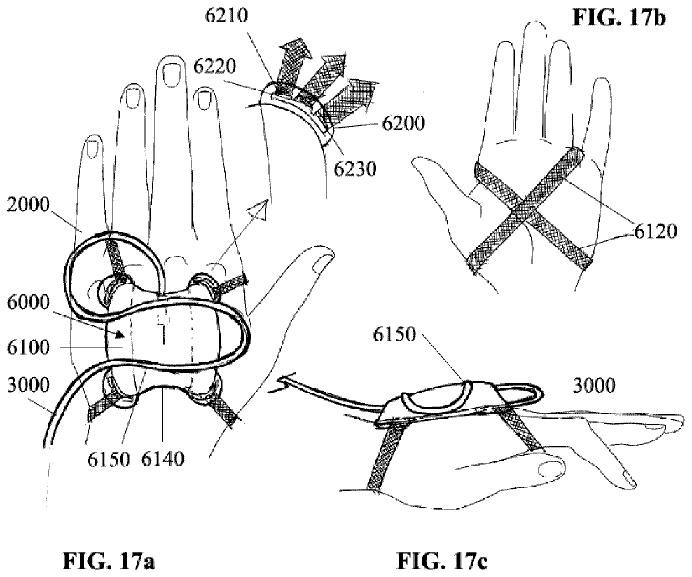

According to a third embodiment as shown in FIGS. 17a, 17b and 17c, an apparatus 6000 may comprise an intravenous line housing 6100 that includes a groove 6150 to hold an intravenous line 3000 by friction fit. The apparatus 6000 may be a glove configured to be suitable for wearing on both a right or left human hand 2000 comprising straps 6120 which may engage with the intravenous line housing 6100 via multi-way adjustable loops such as 3-way adjustable loop (or eyelet) 6200 which may include three different positions 6210, 6220 and 6230 for a strap to pass through. Once the strap 6120 is stretched into place, it may remain in the desired eyelet groove for the particular position. According to an embodiment, the intravenous housing 6100 may also be recessed adjacent the user's wrist for comfort as shown at 6140.

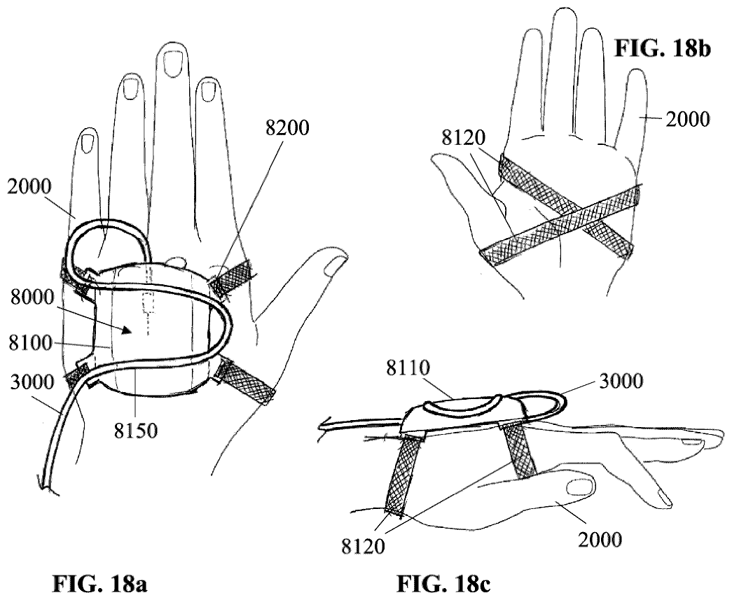

According to a fourth embodiment as shown in FIGS. 18a, 18b and 18c, an apparatus 8000 may comprise an intravenous line housing 8100 that includes a groove 8150 to hold an intravenous line 3000 by friction fit. The apparatus 8000 may be a glove configured to be suitable for wearing on both a right or left human hand 2000 and comprise straps 8120 which may engage with the intravenous line housing 8100 via loops (or eyelets) 8200 for the strap to pass through. Loops (or eyelets) 8200 may be proximate to the perimeter of the intravenous line housing 8100 as shown or elsewhere on the housing 8100 such that the straps 8120 serve to retain the intravenous line housing 8100 on the hand 2000. According to an embodiment, intravenous line housing may comprise a clear injection molded thin wall 8110. According to an embodiment, the intravenous line housing 8100 may have open ends meaning that the housing is not flush to the surface of the skin of a hand 2000 at either end. This may allow space for the intravenous catheter and line beneath the intravenous line housing 8100. According to an embodiment, the intravenous line housing 8100 may have a convex geometry which may allow better articulation of the wrist for extended use.

Various embodiments of the invention have been described in detail. Since changes in and or additions to the above-described best mode may be made without departing from the nature, spirit or scope of the invention, the invention is not to be limited to those details but only by the appended claims.

1. An apparatus for supporting an intravenous line, comprising:
an intravenous line housing defining an intravenous line guide, the intravenous line guide configured to hold the intravenous line and direct the intravenous line along a reversing path and to a substantially proximally-directed entry angle for entry into a dorsal surface of a user's hand; and
a glove secured to the intravenous line housing, the glove configured to hold the intravenous line housing to the user's hand.

2. The apparatus of claim 1, wherein the glove comprises a flexible strap structure; optionally wherein the flexible strap structure is a continuous fabric panel.

3. The apparatus of claim 1, wherein the intravenous line guide comprises a recessed portion in the intravenous line housing.

4. The apparatus of claim 1, wherein the intravenous line housing comprises at least four multi-way adjustable loops or eyelets.

5. The apparatus of claim 4, wherein the multi-way adjustable loops or eyelets are three-way adjustable loops or eyelets.

6. The apparatus of claim 4, wherein the at least four multi-way adjustable loops or eyelets are four three-way adjustable loops or eyelets.

7. The apparatus of claim 2, wherein the flexible strap structure includes a mounting panel and a palm strap, the mounting panel configured to rest on the dorsal surface of the user's hand, the palm strap secured at a first end to a first side of the mounting panel, the palm strap configured to be wrapped around the palm of a user and releasably engaged at a second end to a second side of the mounting panel to hold the mounting panel against the dorsal surface of the user's hand.

8. The apparatus of claim 7, further comprising a releasable fastener secured to one of the second end of the palm strap and second side of the mounting panel, the releasable fastener provided to permit the second end of the palm strap to be releasably engaged with the second side of the mounting panel.

9. The apparatus of claim 8, wherein the releasable fastener is a ladder lock buckle or wherein the flexible strap structure includes a finger loop for engaging a finger of the user's hand.

10. (canceled)

11. (canceled)

12. The apparatus of claim 1, wherein the intravenous line housing includes a set of projections forming an at least one clip for holding the intravenous line as part of the intravenous line guide.

13. The apparatus of claim 12, wherein the intravenous line guide is curved.)

14. The apparatus of claim 13, wherein the curved intravenous line guide includes at least two curvatures, optionally, wherein the curved intravenous line guide is an s-clip guide.

15. (canceled)

16. The apparatus of claim 12, wherein the glove defines an access opening therethrough leaving a portion of the dorsal surface of the user's hand uncovered when the glove is worn, the intravenous line housing configured to direct the entry of the intravenous line into the user's hand through the access opening, optionally wherein the access opening cover is clear to permit a user to view the dorsal surface of the user's hand through the access opening cover.

17. The apparatus of claim 16, wherein the intravenous line housing includes a footing and an access opening cover, the footing secured to the glove and framing the access opening without covering the access opening, the access opening cover configured to be removably secured over the footing to cover the access opening.

18. (canceled)

19. A glove for holding an intravenous line, comprising:
a fabric panel configured to be releasably fastened to a user's hand; and
a set of clips secured to the fabric panel, the set of clips configured to hold the intravenous line and direct the intravenous line along a reversing path and to a substantially proximally-directed entry into a dorsal surface of the user's hand, optionally further comprising an outer cover configured to cover the set of clips and to cover an entry point of the intravenous line.

20. The glove of claim 19, wherein the fabric panel is a continuous panel which defines a clip mounting panel and a palm strap, optionally wherein the fabric panel further defines a finger loop for encircling the base of a finger of the user's hand.

21. (canceled)

22. (canceled)

23. The glove of claim 19, wherein the set of clips are secured to the fabric panel by a set of fasteners, the set of fasteners adhered to the fabric panel and connected to the set of clips via an interference fit.

24. The glove of claim 19, further comprising a releasable fastener secured to a first point on the fabric panel and provided for releasably engaging a second point on the fabric panel.

25. An intravenous line housing defining an intravenous line guide, the intravenous line guide configured to hold the intravenous line and direct the intravenous line along a reversing path and to a substantially proximally-directed entry angle for entry into skin of a patient.

26. An apparatus for supporting an intravenous line comprising the intravenous line housing of claim 25 and a structure secured to the intravenous line housing, the structure configured to secure the intravenous line housing to the patient.
